(19) United States
(12) Patent Application Publication
(10) Pub. No.: US 2016/0220453 A1
(43) Pub. Date: Aug. 4, 2016

(54) ABSORBENT SOAP

(71) Applicant: Kun Yi LIN, Tainan City (TW)

(72) Inventor: Kun Yi LIN, Tainan City (TW)

(21) Appl. No.: 15/013,740

(22) Filed: Feb. 2, 2016

(30) Foreign Application Priority Data
Feb. 2, 2015 (TW) 104103353

(51) Int. Cl. A61K 8/02; A61Q 19/00; A61Q 19/10

(52) U.S. Cl. CPC A61K 8/0233; A61Q 19/10; A61Q 19/007

(57) ABSTRACT

The present disclosure provides an absorbent soap which can absorb water and moisture having multiple cleansing and caring functions to enhance the functions of the conventional bar soaps in terms of dissolving efficiency, moisturizing and fragrance. The absorbent soap includes one or more cleansing-caring layer(s) including cleansing-caring materials and one or more absorbent layer(s) including water-moisture absorbing materials in stack. The stacked structure is formed by combining the cleansing-caring layer(s) and the absorbent layer(s) in a non-specific stacking format. The structure creates residual extra spaces within and between each layer to retain air, so that water from outside can take over the air spaces inside the soap. The water molecule can further and continuously dissolve the cleansing-caring materials in the stacked structure to improve the dissolving efficiency of the cleansing-caring materials.

### BACKGROUND OF THE DISCLOSURE

1. Field of the Invention

The present disclosure relates to absorbent soaps, and specifically relates to an absorbent soap with multiple functions and the method of making the same.

2. Description of Related Art

The bar soap commonly found in the market are used by rubbing the soap onto a user's body with water, so as to convert the solid soap into liquid state. The user can maintain personal hygiene using the liquid soap having cleansing property.

Most of the conventional soap structures are solid bar soap structures, and a few are hollow bar soap structures. With the conventional design of the soap structure, the area of the soap, which comes in contact with water, is only limited to the outer surface of the soap preventing water from penetrating deeply into the soap to dissolve the soap, when the soaps is used. For example, conventionally some soap is made by soaking loofah mesh in liquid soap which later solidifies to form loofah soap. Even though the loofah soap has plenty of pores resulted from filling the loofah mesh, the design of the loofah soap still is not able to efficiently enhance the dissolving rate of the soap. To the contrary, the benefits of the loofah mesh in terms of water-absorbing and foaming enhancement are not utilized in conventional design.

Recently, in order to solve the issue of the dissolving rate of the conventional soap, liquid detergents become replacements. However, for using a liquid detergent, the amount of the liquid soap is difficult to control, which could be wasteful. Regarding efficiency, many liquid detergents are not foamable when applied directly onto the skin, and users sometimes feel that the skin is not washed clean, leaving a slimy feeling. Therefore, when liquid detergents are used, additional foaming tools such as sponge and bath ball are usual companions for foaming to achieve better cleansing efficiency.

The design of the cleansing and caring properties of the conventional soap structures is implemented during the saponification. However, the quality of the design after curation can be limited and affected by the alkaline environment during saponification, which results in the disablement of exhibiting the functions of the added materials by failing to achieve expected effects.

### SUMMARY OF THE DISCLOSURE

The present disclosure provides an absorbent soap which may include water-moisture absorbing materials and cleansing-caring materials to form a stacked structure with multiple properties. The absorbent soap includes at least a cleansing-caring layer which comprises body cleansing-caring materials with easily shapeable property in the shape of powder, granular, liquid, film, sheet, paste, gel, filament or the like; and at least an absorbent layer which comprises water-moisture absorbing materials. The absorbent soap having a stacked structure with multiple functions is formed by combining said cleansing-caring layer and said absorbent layer in a non-specific stacking format. The structure creates extra spaces within and between each of the cleaning-caring layer and absorbent layer to retain air, so that water from outside can take over the air spaces inside the soap, when the soap is used. The water molecule can further and continuously dissolve the cleansing-caring materials in the absorbent soap utilizing a larger tightly-contacted surface. It improves the dissolving efficiency of said cleansing-caring materials and still maintains the functions of the original materials. The present disclosure ultimately solves the problems of the conventional bar soap in terms of dissolving efficiency and maintaining the functions of the original materials through the design of the absorbent soap which is easily shapeable and can be stacked in a non-specific format.

The other advantage of the absorbent soap of the present disclosure is that any combination of the water-moisture absorbing materials and cleansing-caring materials in the finished products can be optionally removed, replaced or added. It enables each material in the soap to exhibit the functions of the original material without affected by the saponification, when the soap is used.

The formed absorbent structure enables a more efficient dissolving reaction to take place between the cleansing-caring material and the water molecules from outside, when the soap is used. It enhances the dissolving efficiency of the cleansing-caring materials and maintains the functions of the original materials.

In order to enable the cleansing-caring layer in the stacked structure of the absorbent soap of the present disclosure to satisfy the stacked structure in a non-specific format and to be squeezed or transformed by external force after the soap is made, the appearance of the cleansing-caring materials may have an easily shapeable property in the shape of powder, granular, liquid, film, sheet, paste, gel, filament, or the like.

The cleansing-caring layer of said absorbent soap may include moisturizing material such as vegetable oils or marine mud; fragrance material such as concentrated fragrance oils or fragrance essences; exfoliation material such as white charcoal (Binchö-tan) particles or fruit kernel powders; cleansing material such as soaps, amino acids, phospholipids, surfactants, or sea salt; or caring material such as hyaluronic acid, collagen, vitamin E or Chinese herbal medicine.

The absorbent layer of said absorbent soap may include water-moisture absorbing materials such as spunlace nonwoven fabric, nonwoven fabric, PET fiber nonwoven fabric, biodegradable fiber nonwoven fabric, cotton, dried plants, sponge, gauze, nylon yarn, fibrous plant, or porous (multiple holes) materials or water-soluble materials.

In order to achieve a more efficient dissolving rate of the absorbent soap, the thickness of the absorbent layer is preferably in the range of 0.01 to 30 mm and the thickness of the cleansing-caring layer is preferably in the range of 0.01 to 8 mm.

### BRIEF DESCRIPTION OF THE DRAWING

FIG. 1 shows a sectional view of the structure of the square absorbent soap of the present disclosure having 1 soap layer and 5 absorbent layers.

FIG. 2 shows a sectional view of the structure of the square absorbent soap of the present disclosure having 2 soap layers, 4 absorbent layers and 1 honey moisturizing layer.

FIG. 3 shows a sectional view of the structure of the concentrically cylindrical absorbent soap of the present disclosure having 1 soap layer and 2 absorbent layers.

FIG. 4 shows a sectional view of the structure of the rectangle absorbent soap of the present disclosure having 1 soap layer, 3 absorbent layers, and 1 caring layer.

FIG. 5 shows a diagram of the appearance of the spiral absorbent soap of the present disclosure.

FIG. 6 shows a sectional view of the structure of the randomly stacked absorbent soap of the present disclosure.

FIG. 7 shows a diagram of the appearance of the vertically stacked absorbent soap of the present disclosure

FIG. 8 shows a diagram of the appearance of the triangle stereo package of the randomly stacked absorbent soap of the present disclosure.

### DETAILED DESCRIPTION OF THE PREFERRED EMBODIMENTS

The following specific examples are used to exemplify the present disclosure. A person of ordinary skills in the art can conceive the other advantages of the present disclosure, based on the disclosure of the specification of the present disclosure. However, these examples are not intended to limit the present disclosure. The present disclosure can also be implemented or applied as described in different specific examples. It is possible to modify and or alter the above examples for carrying out this disclosure by anyone of ordinary skills in the art without contravening its spirit and scope, for different aspects and applications. Therefore, in order to understand the aims, features and effects of the disclosure, the following description will be given of the embodiments of the present invention with accompanying drawings for general disclosure of the present invention.

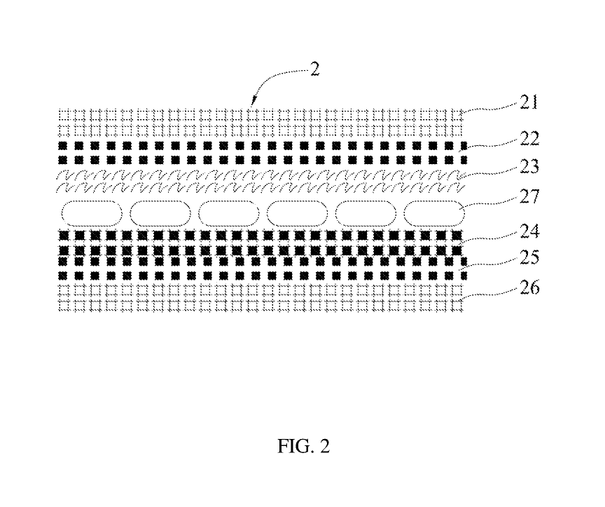

Referring to the embodiment of FIG. 2, the present disclosure provides a design of an absorbent soap having a stacked structure with multiple functions. Said absorbent soap may include one or more layer(s) to react with water: cleansing soap layer 22, soap 25 and honey capsule caring layer 27; and one or more absorbent layer(s) consisting of water-moisture absorbing materials to provide spaces to exchange for the water molecules from outside: spunlace nonwoven fabric 21, dried plant 23, cotton 24, spunlace nonwoven fabric 26. The aforesaid 7 layers were combined together in a square stacked manner vertically from the bottom to the top and then layers 21-26 were secured by sewing to form an absorbent soap having square stacked structure.

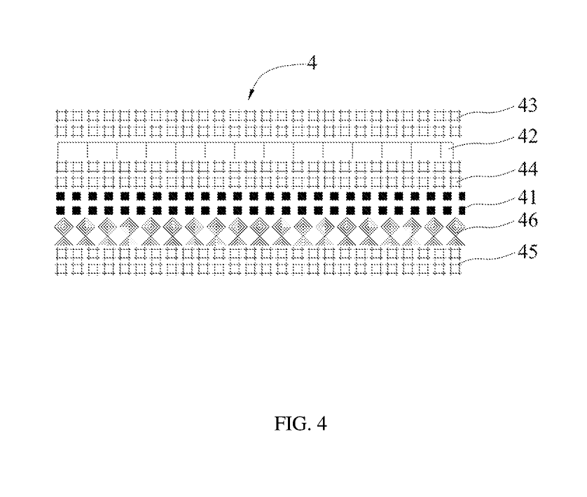

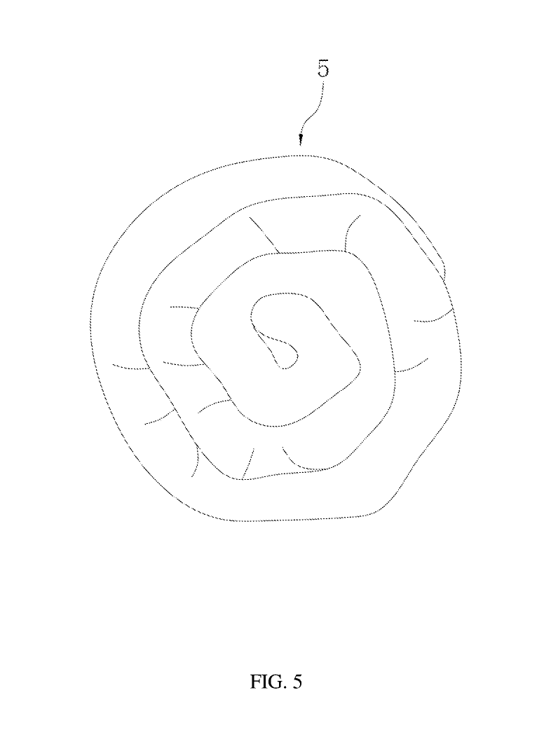

Referring to the embodiments of FIGS. 4 and 5, the present disclosure provides a spirally stacked absorbent soap. Said absorbent soap included: a cleansing layer 41 with the thickness of 3 mm comprising powder soap to react with water; a gel-like caring layer 42 with the thickness of 6 mm comprising grape seed oil, fruit kernel powder, and concentrated rose oil with the functions of moisturizing, exfoliating and fragrant; and one or more layer(s) of absorbent layers (spunlace nonwovens 43, 44, 45 and sponge 46) with the total thickness of 15 mm comprising water-moisture absorbing materials to provide spaces to exchange for water molecules from outside. The aforesaid 6 layers were combined together in a square stacked manner vertically from the bottom to the top. Due to the shapeable property of the stacked structure, the stacked layers were rolled forward and secured with an adhesive to form a spiral absorbent soap having cleansing and caring functions.

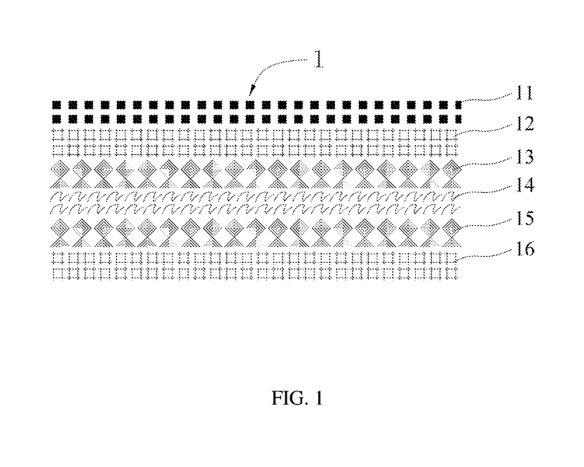

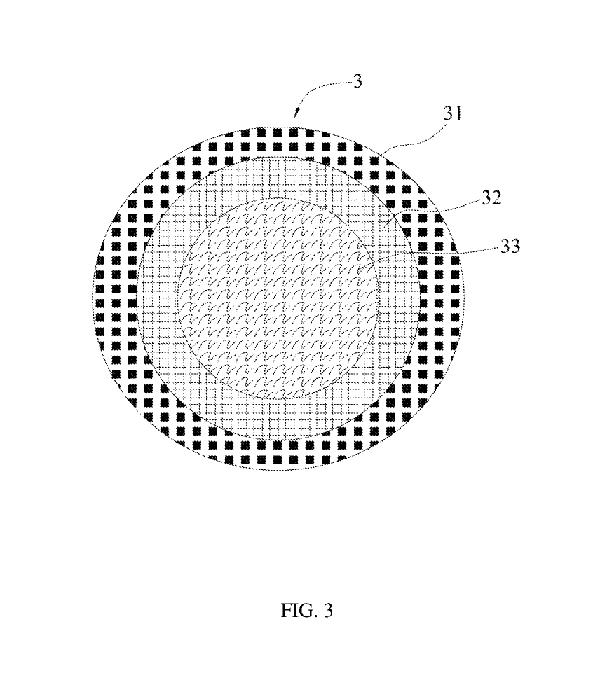

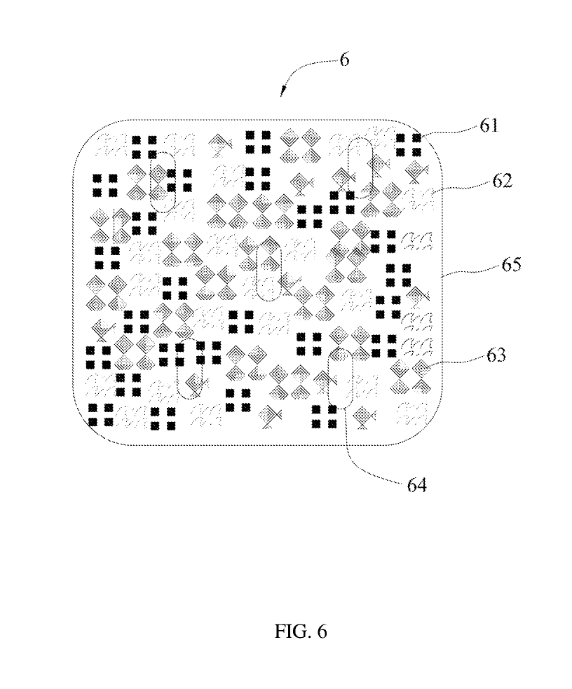

Said absorbent soaps can be designed as the combination of the square stacks shown in FIGS. 1 and 2. In order to provide better visual senses for the users and more efficient dissolving rates of soap, said absorbent soaps can be designed as a concentrically cylindrical absorbent soap as shown in FIG. 3, a randomly stacked absorbent soap shown in FIG. 6, and the absorbent soaps having other stacked structure.

Besides having water-moisture absorbing materials, such as spunlace nonwoven fabric, cotton, or dried plant in the absorbent layer of said absorbent soap, the water-moisture absorbing materials can further be substituted by other water-moisture absorbing materials with same functions, such as sponge, woven fabric, nylon yarn, non-woven fabric, PET fiber nonwoven fabric, biodegradable fiber nonwoven fabric, loofah mesh, Chinese herbal medicines, fibrous plants (such as coconut fibers or rice hulls), porous (multi-holes) materials, water-soluble materials (such as capsules), or the like.

The cleansing layer of said absorbent soap can be substituted with other cleansing materials with same functions other than soap, such as amino acids, phospholipids, surfactants, sea salt or the like.

The caring layer of said absorbent soap may be substituted with moisturizing materials such as vegetable oils, marine mud, honey or the like; or fragrance materials such as concentrated fragrance oils, fragrance essences or the like; or exfoliation materials such as white charcoal (Binchö-tan) particles, kernel powders or the like; or caring materials such as hyaluronic acid, vitamin C, collagen, vitamin E, or the like.

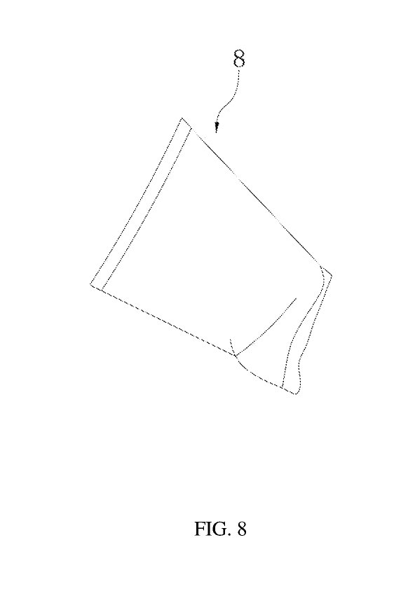

Referring to the embodiments shown in FIGS. 6 and 8, the present disclosure provides an absorbent soap with randomly stacked structure having multiple functions, which was made by the method of: randomly mixing cleansing material soap 61, fragrance material dried lavender 62, absorbent material sponge 63, and vegetable oil capsule 64; and then wrapping the mixture with an absorbent material nonwoven fabric 65. A cleansing-caring solution having the functions of cleansing, fragrant, moisturizing and easy-foaming can be obtained, when said absorbent soap is used.

In the embodiment shown in FIG. 6, after said absorbent soap was made, the nonwoven fabric 65 can be removed, and the contents of sponge 63 and vegetable oil capsule 64 can be taken out replacing with the coconut fiber 23 and the honey capsule 27 shown in FIG. 2 with further addition of the gel-like caring layer 42 (such as grape seed oil, fruit kernel powder or concentrated rose oil as shown in FIG. 4), and then the original nonwoven fabric 65 can be used again as a wrapper to create an absorbent soap with totally different functions.

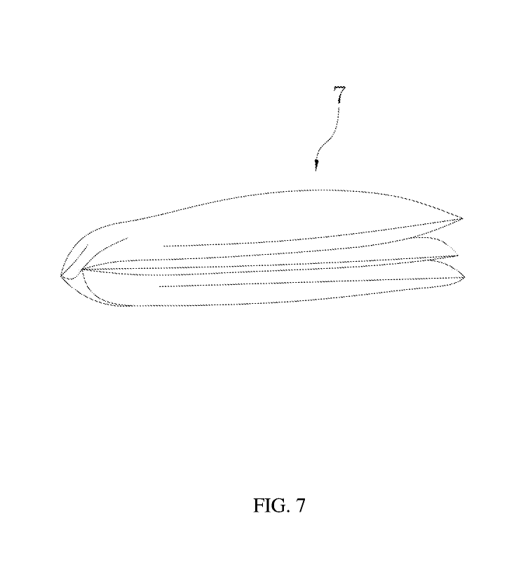

Further referring to the embodiment shown in FIG. 7, the present disclosure provides an absorbent soap with a stacked structure, stacked vertically from the bottom to the top. The 7-layer stacked structure of the absorbent soap shown in FIG. 2 was designed as one unit, and then three of the units were stacked vertically from the top to the bottom, so that the function can be amplified and enhanced.

The example embodiments are described herein in order to explain the principle and the effect of the present disclosure. The examples below should not be construed to limit the scope of the disclosure. It is possible to modify, and or alter the above examples for carrying out this disclosure by anyone of ordinary skills in the art without contravening its spirit and scope. Therefore, the claimed scope of the disclosure depends on the claims attached below.

What is claimed is:

1. An absorbent soap comprises:
at least a cleansing-caring layer comprising materials with functions of body cleansing and caring, which are shapeable depending on requirements for stacking the layers; and
at least an absorbent layer comprising water-moisture absorbing materials,
wherein the at least a cleansing-caring layer and the at least an absorbent layer are combined with each other in stack, to thereby form the absorbent soap for absorbing, cleansing and caring, and the absorbent soap has spaces to retain air within each of the cleaning-caring layer and the absorbent layer and between each of the cleaning-caring layer and the absorbent layer.

2. The absorbent soap of claim 1, wherein the at least a cleansing-caring layer comprises cleansing-caring materials which are shapeable in the shape of powder, granular, liquid, film, sheet, paste, gel, or filament.

3. The absorbent soap of claim 1, wherein the materials of the cleansing-caring layer are selected from the group consisting of moisturizing material, fragrance material, exfoliation material, cleansing material and caring material.

4. The absorbent soap of claim 3, wherein the moisturizing material is selected from the group consisting of vegetable oils, marine mud and honey; the fragrance material is selected from the group consisting of concentrated fragrance oils and fragrance essences; the exfoliation material is selected from the group consisting of white charcoal particles and fruit kernel powders; the cleansing material is selected front the group consisting of soaps, amino acids, phospholipids, surfactants, and sea salt; and the caring material is selected from the group consisting of hyaluronic acid, collagen, and vitamin E.

5. The absorbent soap of claim 1, wherein the at least an absorbent layer comprises absorbent materials of cotton, sponge, woven fabric, nylon yarn, nonwoven fabric, spunlace nonwoven fabric, PET fiber nonwoven fabric, biodegradable fiber nonwoven fabric, loofah mesh, Chinese herbal medicines, capsules, coconut fibers, rice hulls, fibrous plants, dried plants, porous materials, multi-hole materials or water-soluble materials.

6. The absorbent soap of claim 1, wherein the cleaning-caring and absorbent layers are combined in stack by way of vertically stacking, concentrically stacking, spirally stacking or randomly stacking.

7. The absorbent soap of claim 1, wherein the water-moisture absorbing materials or the cleansing-caring materials of the absorbent soap are removable to be replaced or to be added using the water-moisture absorbing materials or cleansing-caring materials of claims 2 to 6, after the absorbent soap is made.

8. The absorbent soap of claim 1, wherein the thickness of the at least an absorbent layer is in the range of 0.01 to 30 mm.

9. The absorbent soap of claim 1, wherein the thickness of the at least a cleansing-caring layer is in the range of 0.01 to 8 mm.
